(19) United States
(12) Patent Application Publication
(10) Pub. No.: US 2025/0026880 A1
(43) Pub. Date: Jan. 23, 2025

(54) POLY(DIALLYLDIMETHYLAMMONIUM) FLUORIDE - A NEW ANTI-CARIES AND REMINERALIZING AGENT

(71) Applicant: The University of Hong Kong, Hong Kong (CN)

(72) Inventors: Chun Hung CHU, Hong Kong (CN); Ying Kei LUNG, Hong Kong (CN)

(21) Appl. No.: 18/754,301

(22) Filed: Jun. 26, 2024

Related U.S. Application Data
(60) Provisional application No. 63/511,792, filed on Jul. 3, 2023.

(51) Int. Cl. C08G 61/12; A61K 6/20; A61K 8/84; A61K 31/787; A61Q 11/00

(52) U.S. Cl. CPC C08G 61/124; A61K 6/20; A61K 8/84; A61K 31/787; A61Q 11/00

(57) ABSTRACT

The subject invention pertains to a novel poly(diallyldimethylammonium) fluoride (PDDAF) compound that provides a restorative material to arrest dental caries, remineralize carious lesions, and inhibit bacterial growth.

### CROSS-REFERENCE TO RELATED APPLICATION

This application claims the benefit of U.S. Ser. No. 63/511,792, filed Jul. 3, 2023, which is hereby incorporated by reference in its entirety including any tables, figures, or drawings.

### BACKGROUND OF THE INVENTION

Dental caries (i.e., dental cavities) are widespread noncommunicable disease, particularly as the consumption of sugar increase worldwide, that result from the acid production of bacteria metabolizing sugars. Severe dental caries affect general health and often causes pain and infection, which may result in tooth extraction.

Poly(diallyldimethylammonium) chloride (PDDACl) is an antibacterial agent. It consists of quaternary ammonium groups (R4N+) that can interact with the negatively charged bacterial cellular membrane by electrostatic attraction. This disrupts the cytoplasmic membranes of bacteria cells and, thus, causes lysis. However, PDDACl does not possess tooth remineralization properties.

Silver diamine fluoride (SDF) has been used to arrest dental caries. It is a non-invasive approach. In 2016, US Food and Drug Administration approved SDF as a drug to treat severe early childhood caries. However, SDF causes permanent, black staining on teeth. Therefore, a novel composition that arrests caries, remineralizes carious lesions, and eliminates the black staining of teeth are urgently needed.

### BRIEF SUMMARY OF THE INVENTION

The subject invention pertains to a novel dental material useful for the management of tooth decay (e.g., dental caries). In certain embodiments, the subject compositions provide a composition comprising poly(diallyldimethylammonium) fluoride (PDDAF) for arresting caries, demineralizing carious lesions, inhibiting bacterial growth, or any combination thereof. In certain embodiments, the PDDAF can be combined with a filler-reinforced resin composite and used for filling of tooth decay. In certain embodiments, silica filler nanoparticles are loaded with PDDAF and added to the resin matrix with mechanical mixing, followed by light curing to prepare the antibacterial and remineralizing dental resin composite.

In certain embodiments, PDDAF is prepared from poly(diallyldimethylammonium) chloride. PDDAF is synthesized from an anion exchange reaction with silver fluoride (PDDACl+AgF=>PDDAF+AgCl). A silver chloride precipitate is formed and can be removed by, for example, filtration or centrifugation. The excess Ag⁺ ions are removed by photo-reduction of silver ions using zinc oxide nanoparticle photo-catalyst. The silver particles formed can be filtered. The PDDAF filtrate is obtained, from which the PDDAF solid is collected by drying in an oven.

In certain embodiments, the subject compositions can be applied to a subject. In certain embodiments, the subject composition can be applied inside the mouth of a subject, including, for example, on the surface of at least 1, 2, 3,4 5, 6, 7,8, 9, 10, 11, 12, 13, 14, 15, 16, 17, 18, 19, 20, 21, 22, 23, 24, 25, 26, 27, 28, 29, 30, 31, 32, or more teeth. In certain embodiments, the tooth has dental caries, carious lesions, or a combination thereof. In certain embodiments, the tooth does not have dental caries nor carious lesions and the subject composition can be used as a prophylactic or preventative treatment. In certain embodiments, the subject compositions can be applied to a subject at least 1, 2, 3, 4, 5, 6, 7, 8, 9, 10, or more times.

### BRIEF DESCRIPTION OF THE DRAWINGS

FIGS. 1A-1B SEM images of (FIG. 1A) poly(diallyldimethylammonium) chloride (PDDACl) and (FIG. 1B) poly(diallyldimethylammonium) fluoride (PDDAF).

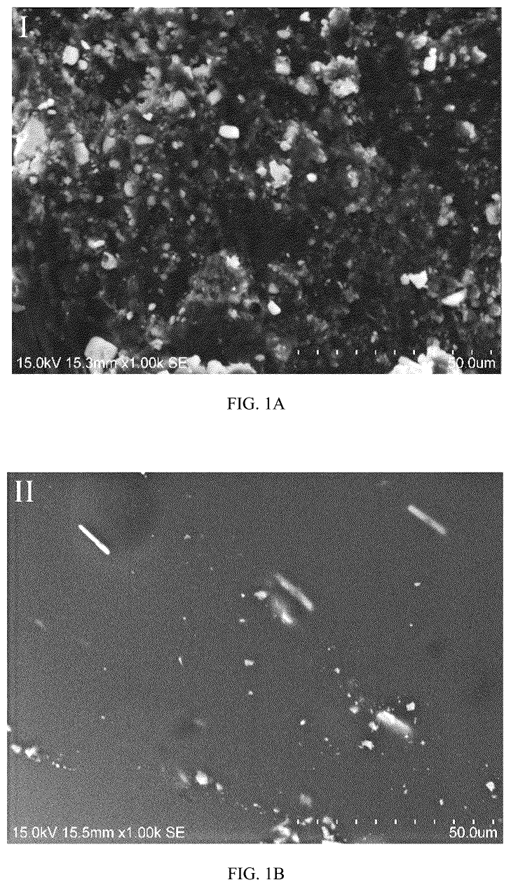

FIGS. 2A-2B EDX spectra of PDDACl (FIG. 2A) and PDDAF (FIG. 2B).

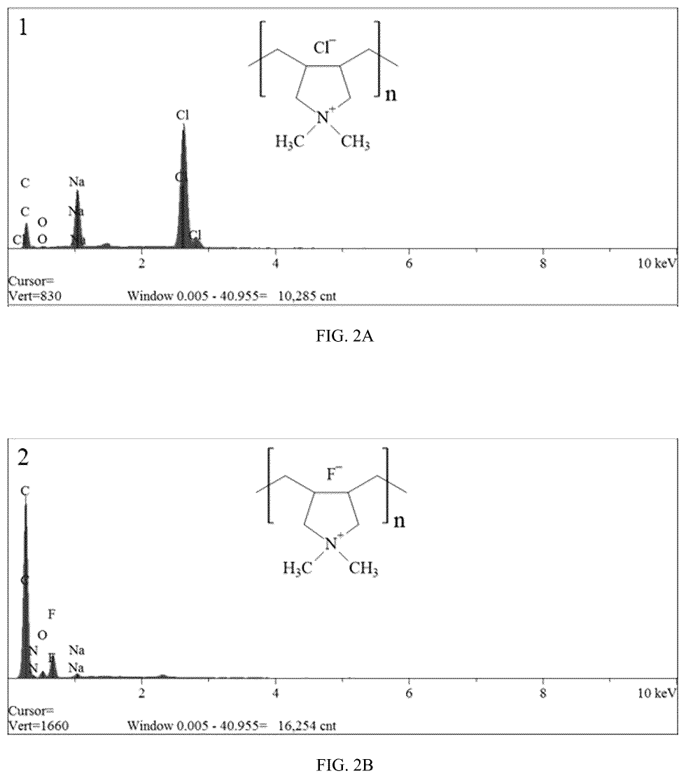

FIG. 3 A calibration plot of fluoride concentration against electric potential of a 280,000 ppm of poly(diallyldimethylammonium) fluoride solution.

FIG. 4 SEM image and EDX spectrum of PDDACl and the chemical structure.

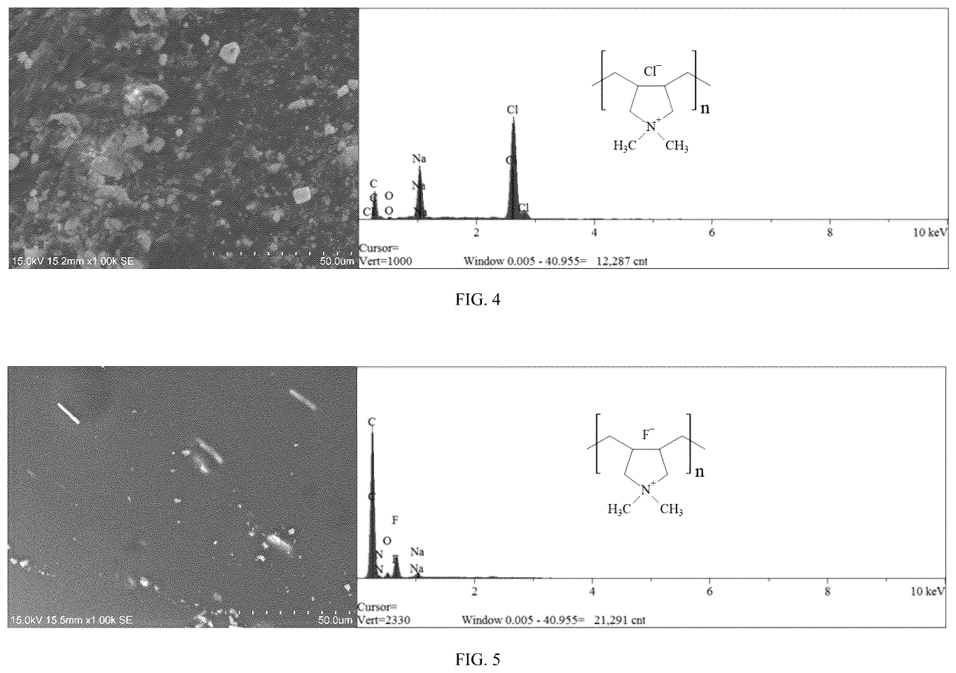

FIG. 5 SEM image and EDX spectrum of PDDAF and the chemical structure.

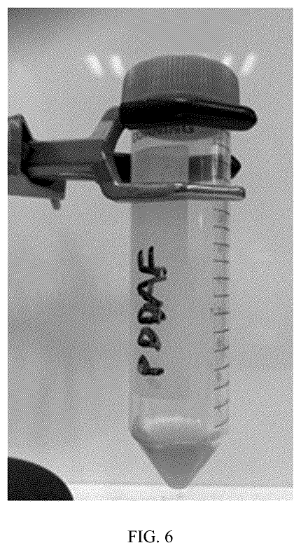

FIG. 6 Poly(diallyldimethylammonium) fluoride (PDDAF) solution.

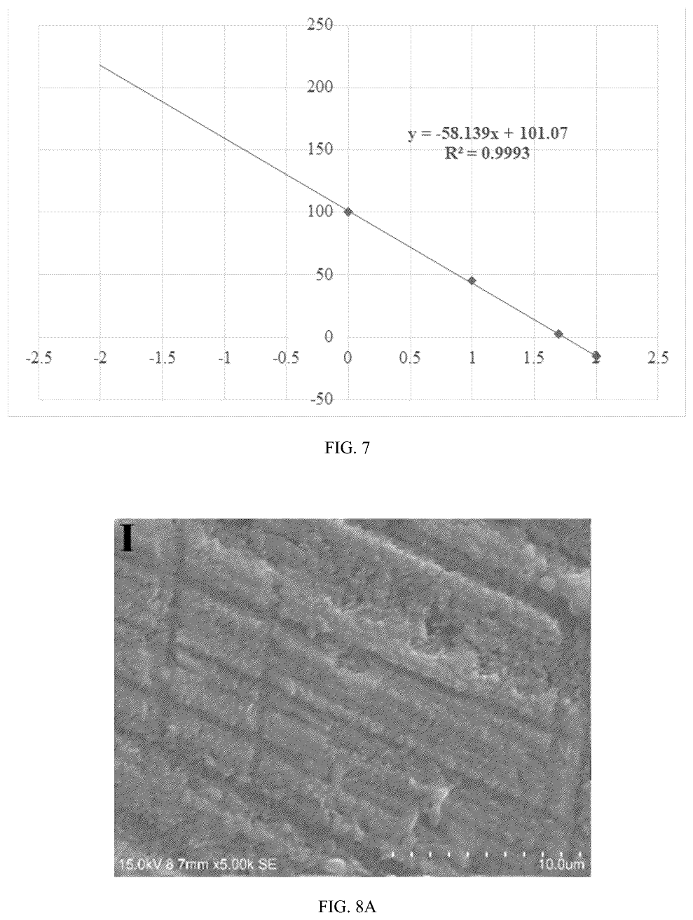

FIG. 7 A calibration plot of fluoride concentration against electric potential of a 350,000 ppm of poly(diallyldimethylammonium) fluoride solution.

FIGS. 8A-8D SEM micrographs (5000×) of dentin specimens: (FIG. 8A) Polished dentin surface, (FIG. 8B) Etched dentin surface by 10% EDTA, (FIG. 8C) Re-mineralized dentin surface with 38% silver diamine fluoride (SDF) soaked in a simulated body fluid for 7 d and (FIG. 8D) Re-mineralized dentin surface with poly(diallyldimethylammonium) fluoride (PDDAF) soaked in a simulated body fluid for 7 d. The volume of both solutions applied on dentin surface is 15 μL.

FIGS. 9A-9D EDX spectra of dentin block surface: (FIG. 9A) Polished dentin surface, (FIG. 9B) Etched dentin surface by 10% EDTA, (FIG. 9C) Re-mineralized dentin surface with 38% silver diamine fluoride (SDF) soaked in a simulated body fluid for 7 d (formation of mineral calcium phosphate and silver chloride) and (FIG. 9D) Re-mineralized dentin surface with poly(diallyldimethylammonium) fluoride (PDDAF) soaked in a simulated body fluid for 7 d (formation of mineral calcium phosphate and calcium fluoride (or fluorapatite)).

FIGS. 10A-10C Agar diffusion test showing zone of inhibition of: FIG. 10A. water; FIG. 10B. PDDAF and FIG. 10C. 38% SDF. Note that the staining of 38% SDF on the paper disc and around the disc after an anaerobic incubation at 37° C. for 24 h. The volume of water, SDF and PDDAF solutions applied is 20 μL.

FIG. 11 Staining effect of PDDAF on demineralized tooth slices after 2 weeks. Negative control: water, positive control: 38% SDF.

FIG. 12 Cell cytotoxicity IC50 of PDDAF and 38% SDF.

### DETAILED DISCLOSURE OF THE INVENTION

### Selected Definitions

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably.

The phrases “consisting essentially of” or “consists essentially of” indicate that the claim encompasses embodiments containing the specified materials or steps and those that do not materially affect the basic and novel characteristic(s) of the claim.

The term “about” means within an acceptable error range for the particular value as determined by one of ordinary skill in the art, which depends in part on how the value is measured, i.e., the limitations of the measurement system. In the context of compositions containing amounts of ingredients where the terms “about” is used, these compositions contain the stated amount of the ingredient with a variation (error range) of 0-10% around the value (X±10%). In other contexts, the term “about” is used provides a variation (error range) of 0-10% around a given value (X±10%). As is apparent, this variation represents a range that is up to 10% above or below a given value, for example, X±1%, X±2%, X±3%, X±4%, X±5%, X±6%, X±7%, X±8%, X±9%, or X±10%.

In the present disclosure, ranges are stated in shorthand to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values. When ranges are used herein, combinations and subcombinations of ranges (e.g., subranges within the disclosed range) and specific embodiments therein are explicitly included.

As used herein, the term “subject” refers to an animal, needing or desiring delivery of the benefits provided by a therapeutic compound. As used herein, the term “animal” may be, for example, humans, pigs, horses, goats, cats, dogs, apes, chimpanzees, orangutans, guinea pigs, hamsters, cows, or sheep. These benefits can include, but are not limited to, the treatment of a health condition, disease, or disorder; prevention of a health condition, disease or disorder; immune health; enhancement of the function of an organ, tissue, or system in the body. The preferred subject in the context of this invention is a human. The subject can be of any age or stage of development, including infant, toddler, adolescent, teenager, adult, or senior.

As used herein, the term “treatment” refers to eradicating, reducing, ameliorating, or reversing a sign or symptom of a health condition, disease, or disorder to any extent, and includes, but does not require, a complete cure of the condition, disease, or disorder. Treating can be curing, improving, or partially ameliorating a disorder. “Treatment” can also include improving or enhancing a condition or characteristic, for example, bringing the function of a particular system in the body to a heightened state of health or homeostasis.

By “reduces” is meant a negative alteration of at least 1%, 5%, 10%, 25%, 50%, 75%, or 100%.

By “increases” is meant as a positive alteration of at least 1%, 5%, 10%, 25%, 50%, 75%, or 100%.

As used herein, an “isolated” or “purified” compound is substantially free of other compounds. In certain embodiments, purified compounds are at least 60% by weight (dry weight) of the compound of interest. Preferably, the preparation is at least 75%, more preferably at least 90%, and most preferably at least 99%, by weight of the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

As used herein, the terms “therapeutically-effective amount,” “therapeutically-effective dose,” “effective amount,” and “effective dose” are used to refer to an amount or dose of a compound or composition thereof that, when administered to a subject, is capable of treating or improving a condition, disease, or disorder in a subject or that is capable of providing enhancement in health or function to an organ, tissue, or body system. In other words, when administered to a subject, the amount is “therapeutically effective.” The actual amount will vary depending on a number of factors including, but not limited to, the particular condition, disease, or disorder being treated or improved; the severity of the condition; the particular organ, tissue, or body system of which enhancement in health or function is desired; the weight, height, age, and health of the patient; and the route of administration.

As used herein, the terms “arresting”, “reducing”, “inhibiting”, “blocking”, “preventing”, “alleviating”, or “relieving” when referring to a compound, mean that the compound brings down the occurrence, severity, size, volume or associated symptoms of dental caries and/or carious lesions by at least about 7.5%, 10%, 12.5%, 15%, 17.5%, 20%, 22.5%, 25%, 27.5%, 30%, 35%, 40%, 45%, 50%, 55%, 60%, 65%, 70%, 75%, 80%, 90%, or 100% compared to how dental caries and/or carious lesions would normally exist without application of the compound or a composition comprising the compound.

As used herein, the terms “dental caries”, “tooth decay”, or “dental cavities” to a disease that involves the deterioration of the structure of a tooth involving a series of demineralization/neutrality/remineralization of dental hard tissue in primary and permanent dentition. Dental caries are often caused by the acid produced by bacteria through the anaerobic metabolism of sugars and fermentable carbohydrates in the mouth. The acids produced dissolve the hydroxyapatite of dental hard tissues, i.e., demineralization. The remineralization process is a repair mechanism to restore the hydroxyapatite crystal lattice. When the rate of demineralization is faster than remineralization, this leads to dental caries.

As used herein, the term “carious lesion” is a visible macroscopic breakdown in the hard tooth structure resulting from the process of dental caries. The surface or hole and the area may have softened walls or floor.

In some embodiments of the invention, the method comprises administration of multiple doses of the compositions of the subject invention. The method may comprise administration of therapeutically effective doses of a composition comprising the compound or composition thereof of the subject invention as described herein once a week, once a month, once a quarter, twice a year, once a year, or a lower frequency. Moreover, treatment of a subject with a therapeutically effective amount of the compositions of the invention can include a single treatment or can include a series of treatments. It will also be appreciated that the effective dosage of a compound or composition thereof used for treatment may increase or decrease over the course of a particular treatment. Changes in dosage may result and become apparent from the results of diagnostic assays determine the presence (or absence) or dental caries and/or carious lesions, which are known in the art. Specifically, the identification of dental caries and/or carious lesions includes, for example, intraoral radiographs.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Other features and advantages of the invention will be apparent from the following description of the preferred embodiments thereof, and from the claims. All references cited herein are hereby incorporated by reference.

### Poly(Diallyldimethylammonium) Fluoride Compositions

Provided herein are cationic polymer fluoride compounds that can treat dental caries, carious lesions, or a combination thereof.

In certain embodiments, the compound of the subject invention is (poly(diallyldimethylammonium) fluoride) according to formula (I):

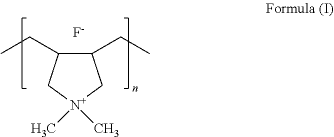

wherein n is about 50 to about 10000, about 53 to about 3092, about 100 to about 1855, or about 1484.

In certain embodiments, the compound of the subject invention is (poly(diallyldimethylammonium) fluoride) according to Formula (II):

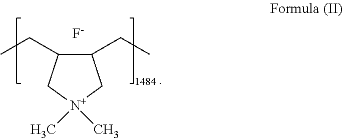

In one embodiment, the subject compositions are formulated as an orally-consumable product, such as, for example a food item, capsule, pill, or liquid. An orally consumable pharmaceutical is any physiologically active substance delivered via initial direct application to an oral surface, preferably the surface of a tooth.

Orally-consumable products according to the invention are any preparations or compositions suitable for consumption, for nutrition, for oral hygiene, or for pleasure, and are products intended to be introduced into the human or animal oral cavity, to remain there for a certain period of time, and then either be swallowed (e.g., food ready for consumption or pills) or to be removed from the oral cavity again (e.g., chewing gums or products of oral hygiene or medical mouth washes or gels). While an orally-deliverable pharmaceutical can be formulated into an orally consumable product, and an orally consumable product can comprise an orally deliverable pharmaceutical, the two terms are not meant to be used interchangeably herein.

Orally-consumable products include all substances or products intended to be ingested by humans or animals in a processed, semi-processed, or unprocessed state. This also includes substances that are added to orally consumable products (particularly food and pharmaceutical products) during their production, treatment, or processing and intended to be introduced into the human or animal oral cavity.

Orally-consumable products can also include substances intended to be swallowed by humans or animals and then digested in an unmodified, prepared, or processed state; the orally consumable products according to the invention therefore also include casings, coatings, or other encapsulations that are intended to be swallowed together with the product or for which swallowing is to be anticipated.

In one embodiment, the orally-consumable product is a capsule, pill, syrup, emulsion, or liquid suspension containing a desired orally deliverable substance. In one embodiment, the orally-consumable product can comprise an orally deliverable substance in powder form, which can be mixed with water or another liquid to produce a drinkable orally-consumable product.

In some embodiments, the orally-consumable product according to the invention can comprise one or more formulations intended for nutrition or pleasure. These particularly include baking products (e.g., bread, dry biscuits, cake, and other pastries), sweets (e.g., chocolates, chocolate bar products, other bar products, fruit gum, coated tablets, hard caramels, toffees and caramels, and chewing gum), alcoholic or non-alcoholic beverages (e.g., cocoa, coffee, green tea, black tea, oolong tea, white tea, black or green tea beverages enriched with extracts of green or black tea, Rooibos tea, Honeybush tea, other herbal teas, fruit-containing lemonades, isotonic beverages, soft drinks, nectars, fruit and vegetable juices, and fruit or vegetable juice preparations), instant beverages (e.g., instant cocoa beverages, instant tea beverages, and instant coffee beverages), meat products (e.g., ham, fresh or raw sausage preparations, and seasoned or marinated fresh meat or salted meat products), eggs or egg products (e.g., dried whole egg, egg white, and egg yolk), cereal products (e.g., breakfast cereals, muesli bars, and pre-cooked instant rice products), dairy products (e.g., whole fat or fat reduced or fat-free milk beverages, rice pudding, yoghurt, kefir, cream cheese, soft cheese, hard cheese, dried milk powder, whey, butter, buttermilk, and partly or wholly hydrolyzed products containing milk proteins), products from soy protein or other soy bean fractions (e.g., soy milk and products prepared thereof, beverages containing isolated or enzymatically treated soy protein, soy flour containing beverages, preparations containing soy lecithin, fermented products such as tofu or tempeh products prepared thereof and mixtures with fruit preparations and, optionally, flavoring substances), fruit preparations (e.g., jams, fruit ice cream, fruit sauces, and fruit fillings), vegetable preparations (e.g., ketchup, sauces, dried vegetables, deep-freeze vegetables, pre-cooked vegetables, and boiled vegetables), snack articles (e.g., baked or fried potato chips (crisps) or potato dough products and extrudates on the basis of maize or peanuts), products on the basis of fat and oil or emulsions thereof (e.g., mayonnaise, remoulade, and dressings), other ready-made meals and soups (e.g., dry soups, instant soups, and pre-cooked soups), seasonings (e.g., sprinkle-on seasonings), sweetener compositions (e.g., tablets, sachets, and other preparations for sweetening or whitening beverages or other food). The present compositions may also serve as semi-finished products for the production of other compositions intended for nutrition or pleasure.

The subject composition can further comprise one or more pharmaceutically acceptable carriers, and/or excipients, and can be formulated into preparations, for example, solid, semi-solid, liquid, or gaseous forms, such as tablets, capsules, powders, granules, ointments, solutions, suppositories, injections, inhalants, and aerosols.

In certain embodiments, the PDDAF can be used to prepare an antibacterial and remineralizing hydrogel with carboxymethyl cellulose (CMC) and a cross-linking agent, such as, for example, citric acid.

In certain embodiments, the PDDAF can be encapsulated in poly (methylmethacrylate) (PMMA) nanoparticles.

In certain embodiments, the PDDAF can be mixed with a methylmethacrylate (MMA) solution and azobisisobutyronitrile (AIBN) initiator.

In certain embodiments, the PDDAF can be combined with a filler-reinforced resin composite and used for filling of tooth decay. In certain embodiments, silica filler nanoparticles are loaded with PDDAF and added to the resin matrix with mechanical mixing, followed by light curing to prepare the antibacterial and remineralizing dental resin composite.

The term “pharmaceutically acceptable” as used herein means compatible with the other ingredients of a pharmaceutical composition and not deleterious to the recipient thereof.

Carriers and/or excipients according the subject invention can include any and all solvents, diluents, buffers (such as, e.g., neutral buffered saline, phosphate buffered saline, or optionally Tris-HCl, acetate or phosphate buffers), oil-in-water or water-in-oil emulsions, aqueous compositions with or without inclusion of organic co-solvents suitable for, e.g., IV use, solubilizers (e.g., Polysorbate 65, Polysorbate 80), colloids, dispersion media, vehicles, fillers, chelating agents (e.g., EDTA or glutathione), amino acids (e.g., glycine), proteins, disintegrants, binders, lubricants, wetting agents, emulsifiers, sweeteners, colorants, flavorings, aromatizers, thickeners (e.g. carbomer, gelatin, or sodium alginate), coatings, preservatives (e.g., Thimerosal, benzyl alcohol, polyquaterium), antioxidants (e.g., ascorbic acid, sodium metabisulfite), tonicity controlling agents, absorption delaying agents, adjuvants, bulking agents (e.g., lactose, mannitol) and the like. The use of carriers and/or excipients in the field of drugs and supplements is well known. Except for any conventional media or agent that is incompatible with the target health-promoting substance or with the composition, carrier or excipient use in the subject compositions may be contemplated.

The subject invention also concerns kits comprising in one or more containers of at least 1, 2, 3, 4, 5, 6, 7, 8, 9, 10 or more compounds of the subject invention. A kit of the invention can also comprise one or more compounds, biological molecules, or drugs. In one embodiment, a kit of the invention comprises a compound of the subject invention.

The invention further provides kits, including compounds of the subject invention and pharmaceutical formulations, packaged into suitable packaging material, optionally in combination with instructions for using the kit components, e.g., instructions for performing a method of the invention. In one embodiment, a kit includes an amount of compounds of the subject invention and instructions for administering the compounds of the subject invention to a subject in need of treatment on a label or packaging insert. In further embodiments, a kit includes an article of manufacture, for delivering the compounds of the subject invention into a subject locally, regionally, or systemically, for example.

As used herein, the term “packaging material” refers to a physical structure housing the components of the kit. The packaging material can maintain the components sterilely and can be made of material commonly used for such purposes (e.g., paper, corrugated fiber, glass, plastic, foil, ampules, etc.). The label or packaging insert can include appropriate written instructions, for example, practicing a method of the invention, e.g., treating dental caries, carious lesions, or a combination thereof, an assay for identifying a subject having dental caries, carious lesions, or a combination thereof, etc. Thus, in additional embodiments, a kit includes a label or packaging insert including instructions for practicing a method of the invention in solution, in vitro, in vivo, or ex vivo.

Instructions can, therefore, include instructions for practicing any of the methods of the invention described herein. For example, pharmaceutical compositions can be included in a container, pack, or dispenser together with instructions for administration to a subject to treat dental caries or carious lesions. Instructions may additionally include appropriate administration route, dosage information, indications of a satisfactory clinical endpoint or any adverse symptoms that may occur, storage information, expiration date, or any information required by regulatory agencies such as the Food and Drug Administration or European Medicines Agency for use in a human subject.

The instructions may be on “printed matter,” e.g., on paper or cardboard within the kit, on a label affixed to the kit or packaging material, or attached to a vial or tube containing a component of the kit. Instructions may comprise voice or video tape and additionally be included on a computer readable medium, such as a disk (hard disk), optical CD such as CD- or DVD-ROM/RAM, Blu-Ray Disc™, electrical storage media such as RAM and ROM and hybrids of these such as magnetic/optical storage media.

Kits can additionally include a buffering agent, a preservative, or an agent for stabilizing the compounds of the subject invention. Each component of the kit can be enclosed within an individual container or in a mixture and all of the various containers can be within single or multiple packages.

Methods of Treating Dental Caries and/or Carious Lesions

In certain embodiments, the compounds of the subject invention can be used for treating dental caries, carious lesions, or a combination thereof in a subject. In certain embodiments, the cationic polymer backbone of the subject compounds inhibit bacterial growth and the fluoride of the subject compounds remineralizes teeth. The underlying principle of antibacterial action of cationic polymer of the subject compounds is through an electrostatic attraction to the bacterial cell membrane. The cationic polymer of the subject compounds contains positively charged group, ammonium group, and the bacterial cell membranes are negatively charged. The cationic polymer of the subject compounds is attracted to the bacterial cell membrane by the electrostatic attraction. This is followed by penetration into the bacterial cytoplasm and insertion into the phospholipid bilayer. This disrupts the cytoplasmic membranes and causes lysis.

Fluoride ions diffuse into the enamel surface and are adsorbed into its surface. It enhances tooth remineralization by attracting calcium and phosphate ions together and forming of fluoroapatite crystals on the demineralized enamel surface in the carious lesion. The fluoroapatite is much more resistant to acid dissolution than hydroxyapatite and carbonated apatite. As a result, the new mineral formed prevents dental caries.

In certain embodiments, the subject compositions can be applied orally to the subject. In certain embodiments, the application comprises rinsing, spraying, dropping, brushing, injecting, or any combination thereof to the oral cavity of the subject, preferably to the surface of a tooth. In certain embodiments, the subject compounds can be administered to a subject before the subject is diagnosed with dental caries, carious lesions, or a combination thereof or to treat a subject diagnosed with dental caries, carious lesions, or a combination thereof. In certain embodiments, the subject is a mammal. In preferred embodiments, the mammal is a human.

In certain embodiments, the compounds and/or compositions of subject invention can be used in methods of treating dental caries, carious lesions, or a combination thereof.

In preferred embodiments, the cationic polymer fluoride is designed to arrest caries, remineralize carious lesions, and eliminate black staining formation. PDDAF is more photo stable than SDF towards staining formation. PDDAF does not form precipitate in the oral environment as SDF, e.g., formation of silver chloride. Silver chloride is not photo stable and forms black staining. This eliminates the black staining on teeth after application. Therefore, application of PDDAF results in good aesthetic outcomes. PDDAF is an antibacterial organic polymer fluoride, which is different from the antibacterial inorganic SDF in terms of chemical structures and antibacterial mechanism of action.

Therapeutic or prophylactic application of the subject compounds and compositions containing the compounds thereof, can be accomplished by any suitable therapeutic or prophylactic method and technique presently or prospectively known to those skilled in the art. The compounds can be administered by any suitable route known in the art including, for example, oral or intravascular (e.g., intravenous) routes of administration. In preferred embodiments, the compound or composition thereof can be administered orally. Administration of the compounds of the invention can be continuous or at distinct intervals as can be readily determined by a person skilled in the art.

In some embodiments, an amount of the compounds can be administered 1 time per day, for 1, 2, 3, 4, 5, 6, 7, or more days. Treatment can continue as needed, e.g., for several weeks, for several months, for several years, for several decades. For example, in some embodiments, an initial application of the composition containing a concentration of the subject compounds (e.g., PDDAF) of about 1% to about 99%, about 5% to about 50%, about 12% to about 38%, or about 30%. To provide for the administration of such concentrations for the desired therapeutic treatment, pharmaceutical compositions of the invention will advantageously comprise between about 0.1% and 99%, and especially, 1% and 40% by weight of the total of one or more of the compounds based on the weight of the total composition including carrier or diluent.

### Materials and Methods

PDDAF is prepared from poly(diallyldimethylammonium) chloride. The PDDAF is synthesized from an anion exchange reaction with silver fluoride (PDDACl+AgF=>PDDAF+AgCl). A silver chloride precipitate is formed and can be removed by filtration. The excess Ag⁺ ions are removed by photo-reduction of silver ions using zinc oxide nanoparticle photo-catalyst. The silver particles formed are filtered. The PDDAF filtrate is obtained, from which the PDDAF solid is collected by drying in an oven.

PDDAF can be prepared from poly(diallyldimethylammonium) chloride, PDDACl, and silver fluoride by the anion exchange reaction.

In an example, the synthesis of PDDAF was carried out in an aqueous solution. PDDACl (0.95 g) was added to 5 ml deionized water. The mixture was heated to 50° C. with stirring until the PDDACl was dissolved completely. Silver fluoride (0.8 g) was dissolved in 5 ml deionized water. The silver fluoride solution was added drop by drop to the PDDACl solution. A white precipitate was formed. The silver fluoride solution was continuously added until no more white precipitate was formed. The white precipitate was removed by centrifugation.

The excess silver ions were removed by photo-reduction using zinc oxide nanoparticles (ZnO NPs) photo-catalyst. ZnO NPs (0.1 g) was added to the supernatant fluid. The mixture was stirred for 15 mins. Then, the mixture was UV irradiated at 365 nm for 24 h. The black silver precipitate formed was removed by centrifugation. The supernatant fluid of PDDAF was obtained. The PDDAF solid was collected by drying in an oven. The PDDAF solution was prepared by dissolving the PDDAF solid in water at 37° C. for 24 h. The pH of the solution was adjusted to 9-11 by potassium hydroxide.

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### Example 1—Fluoride Ion Concentration Measurement of the Cationic Polymer Fluoride

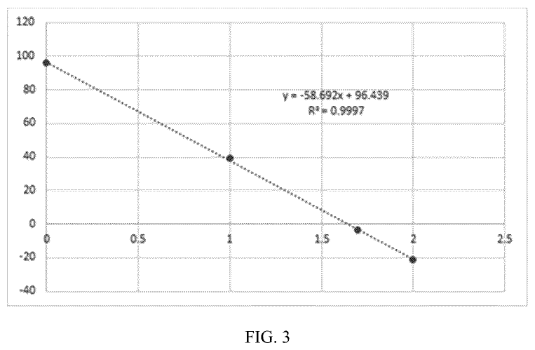

The F ion concentration of a 280,000 ppm of poly(diallyldimethylammonium) fluoride solution was measured using a fluoride ion selective electrode which was connected to a potentiometer. Calibration of the fluoride electrode was performed using standardized fluoride solutions with concentrations at 100, 50, 10 and 1 ppm, which was buffered using equal volume of total ionic strength adjustment buffer. A calibration plot of log [F⁻] against electric potential was obtained (FIG. 3).

The PDDAF solution was diluted 500 times and then the F ion concentration was measured. Three measurements were made. The mean fluoride ion concentration was 41783.33±353.2 ppm.

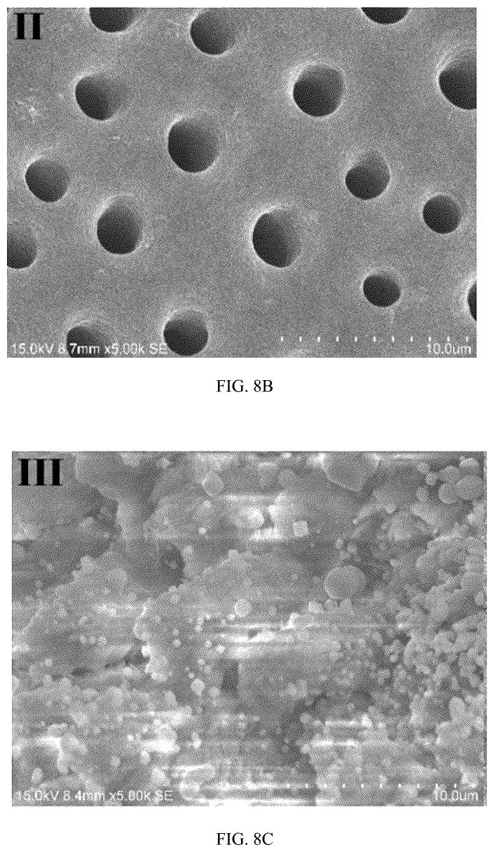

The F ion concentration of a 350,000 ppm of poly(diallyldimethylammonium) fluoride solution was measured using a fluoride ion selective electrode, which was connected to a potentiometer. Calibration of the fluoride electrode was performed using standardized fluoride solutions, which was buffered using equal volume of total ionic strength adjustment buffer. A calibration plot of log [F⁻] against electric potential was obtained (FIG. 7).

The measured F ion concentrations (three samples):

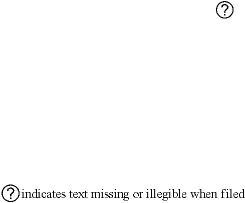

As a result, the F ion concentration of the 350, 000 ppm PDDAF solution=measured [F⁻]×400 (diluted for measurement):

| Sample | F/ppm |
|---|---|
| 1 | 51320 |
| 2 | 53816 |
| 3 | 58024 |

The mean F ion concentration of the 350,000 ppm of poly(diallyldimethylammonium) fluoride solution=54,386.67±2766.48 ppm.

### Example 2—Remineralizing Properties of the Cationic Polymer Fluoride

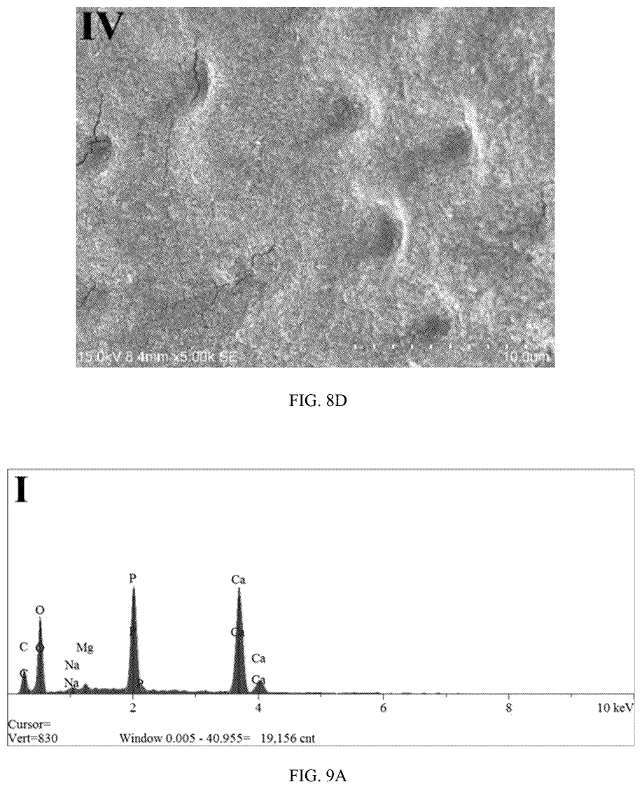

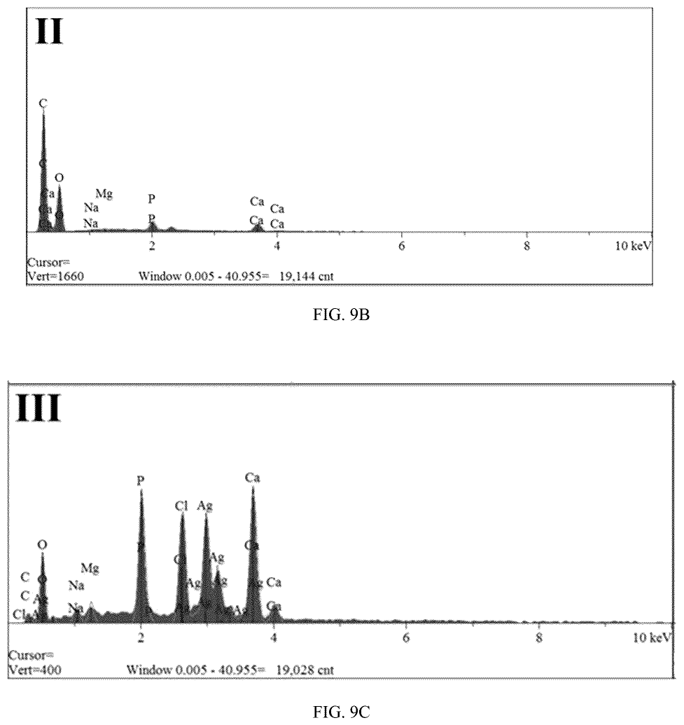

The remineralizing properties of 280,000 ppm cationic polymer fluoride solution was examined. The concentration of 38% silver diamine fluoride is 300,000 ppm with respect to silver fluoride. Dentin blocks with the dimensions of 4 mm×4 mm×2 mm were prepared (FIGS. 8C-8D, 9C-9D).

### Example 3—Antibacterial Activity of the Cationic Polymer Fluoride

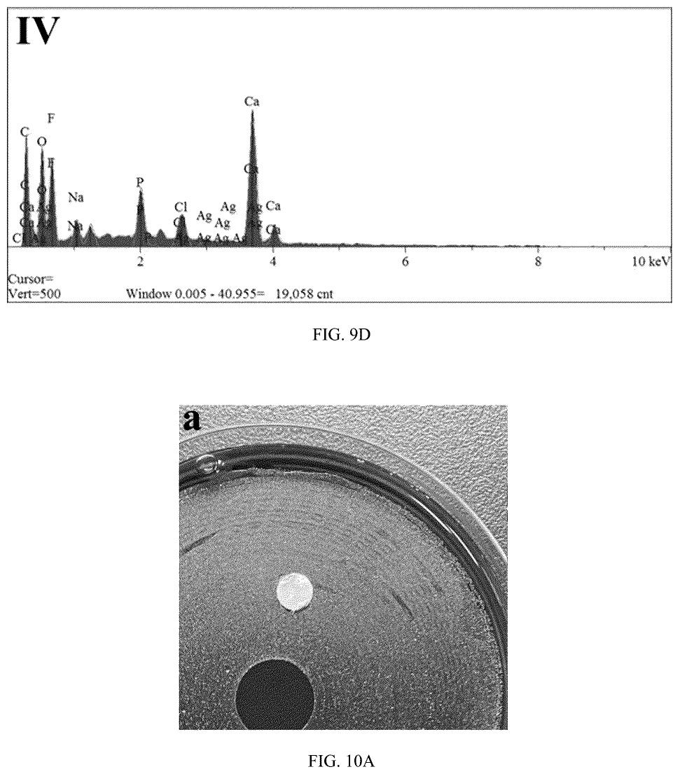

The antibacterial activity of the cationic polymer fluoride is evaluated against Streptococcus mutans using the agar diffusion test. The positive group is 38% silver diamine fluoride (300,000 ppm with respect to AgF). The negative group is water. The concentration of the cationic polymer fluoride is 280,000 ppm (FIGS. 10A-10C).

### Example 4—Staining Effect of the Cationic Polymer Fluoride

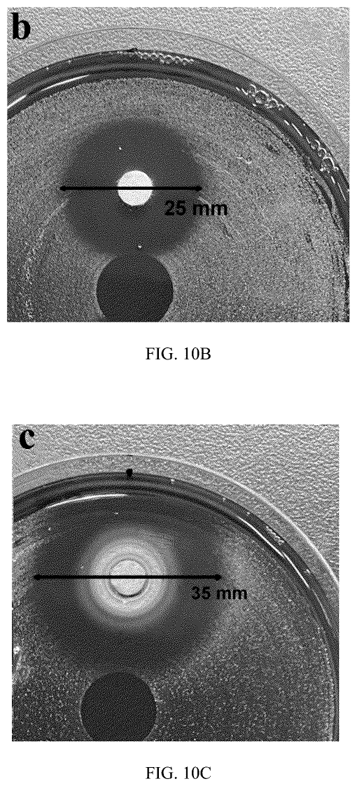

We evaluated the staining effect of the 280,000 ppm PDDAF solution on demineralized tooth slices. We applied 25 μL of the PDDAF solution on the demineralized tooth slice. The control groups were water and 38% silver diamine fluoride (SDF). We found that 38% SDF stained the demineralised tooth slices black, whereas the PDDAF solution did not discolor the tooth slices after 2 weeks FIG. 11).

### Example 5—Cell Cytotoxic Test

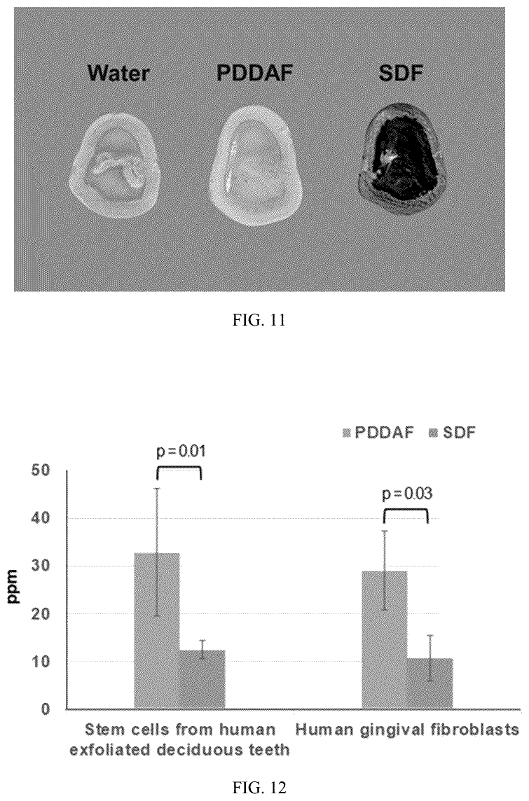

We examined the cell cytotoxicity on 280,000 ppm PDDAF by using human gingival fibroblast cells (HGF-1) and stem cells from human exfoliated deciduous teeth (SHED1). FIG. 12 shows the IC50 values measured for PDDAF and 38% silver diamine fluoride (SDF). The cell cytotoxicity of PDDAF to HGF-1 and SHED1 cells was significantly lower than that of SDF. The results showed that PDDAF is safe for human gingival fibroblasts and dental pulp cells.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### Exemplary Embodiments

EMBODIMENT 1. A compound of formula (I), wherein the compound is poly(diallyldimethylammonium) fluoride (PDDAF):

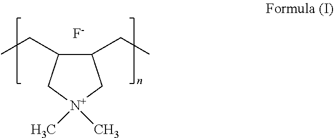

wherein n is about 50 to about 10000.

EMBODIMENT 2. The compound of embodiment 1, wherein n is 1484, according to Formula II:

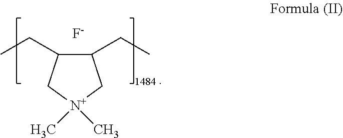

EMBODIMENT 3. A composition comprising the compound of embodiment 1, or a pharmaceutically acceptable salt thereof, and a pharmaceutically acceptable carrier or excipient.

EMBODIMENT 4. A method of treating dental caries, carious lesions, or a combination thereof in a subject, the method comprising:
providing a composition comprising a compound of formula (I), wherein the compound is poly(diallyldimethylammonium) fluoride (PDDAF):

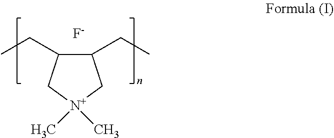

wherein n is about 50 to about 10000; and
applying the composition orally to the subject.

EMBODIMENT 5. The method of embodiment 4, wherein n is 1484.

EMBODIMENT 6. The method of embodiment 4, wherein the subject is a mammal.

EMBODIMENT 7. The method of embodiment 6, wherein the mammal is a human.

EMBODIMENT 8. A method of treating dental caries, carious lesions, or a combination thereof in a subject, the method comprising applying an affective amount of the composition of embodiment 3 to a tooth of the subject.

EMBODIMENT 9. The method any preceding embodiment, wherein applying the composition to the tooth of the subject comprises rinsing, spraying, dropping, brushing, injecting, or any combination thereof to the subject.

EMBODIMENT 10. The method of any preceding embodiment, wherein the subject has dental caries.

EMBODIMENT 11. The method of any preceding embodiment, wherein the subject has a carious lesion.

EMBODIMENT 12. The method of any preceding embodiment, wherein the subject has a dental caries and a carious lesion.

EMBODIMENT 13. The method of any preceding embodiment, wherein the composition is applied to the subject before the subject is diagnosed with dental caries, carious lesions, or a combination thereof.

EMBODIMENT 14. The method of any preceding embodiment, wherein the compound of formula (I) inhibits bacterial growth in the subject.

EMBODIMENT 15. The method of any preceding embodiment, wherein the compound of formula (I) remineralizes a tooth of the subject.

EMBODIMENT 16. The method of any preceding embodiment, wherein an effective amount of the composition is applied orally to the subject as a single treatment.

EMBODIMENT 17. The method of any of embodiments 4-15, wherein an effective amount of the composition is applied orally to the subject as a series of treatments.

EMBODIMENT 18. A kit for treating a subject with at least one of a dental caries or a carious lesion, comprising the composition of embodiment 3 and, optionally, a buffering agent, a preservative, or an agent for stabilizing the compound.

EMBODIMENT 19. The kit of embodiment 18, wherein the kit is packaged into suitable packaging material and comprises instructions for administering the compound to a subject.

EMBODIMENT 20. The kit of embodiment 19, wherein the instructions comprise one or more of paper or cardboard within the kit, label affixed to the kit, voice or video tape, or computer readable medium selected for the group consisting of disk, optical CD, DVD-ROM/RAM, Blu-Ray Disc™, electrical storage media comprising RAM and ROM, and any combination thereof.

### REFERENCES

Synthesis and characterization of an anti-caries and remineralizing fluorine-containing cationic polymer PHMB-F. Biomaterials Science 2020; 9: doi:10.1039/DOBM01627F.

US Patent; Patent No: U.S. Pat. No. 9,156,772 B2 (Fluoride-releasing compositions)—2015

We claim:

1. A compound of formula (I), wherein the compound is poly(diallyldimethylammonium) fluoride (PDDAF): 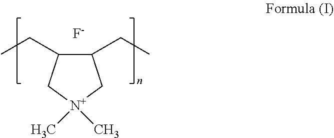
wherein n is about 50 to about 10000.

2. The compound of claim 1, wherein n is 1484, according to Formula II:

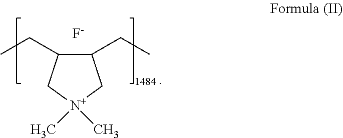

3. A composition comprising the compound of claim 1, or a pharmaceutically acceptable salt thereof, and a pharmaceutically acceptable carrier or excipient.

4. A method of treating dental caries, carious lesions, or a combination thereof in a subject, the method comprising:
providing a composition comprising a compound of formula (I), wherein the compound is poly(diallyldimethylammonium) fluoride (PDDAF): 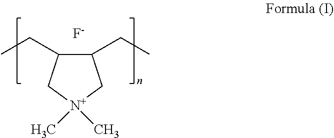
wherein n is about 50 to about 10000; and
applying the composition orally to the subject.

5. The method of claim 4, wherein n is 1484.

6. The method of claim 4, wherein the subject is a mammal.

7. The method of claim 6, wherein the mammal is a human.

8. A method of treating dental caries, carious lesions, or a combination thereof in a subject, the method comprising applying an effective amount of the composition of claim 3 to a tooth of the subject.

9. The method of claim 4, wherein applying the composition to the tooth of the subject comprises rinsing, spraying, dropping, brushing, injecting, or any combination thereof to the subject.

10. The method of claim 4, wherein the subject has dental caries.

11. The method of claim 4, wherein the subject has a carious lesion.

12. The method of claim 4, wherein the subject has a dental caries and a carious lesion.

13. The method of claim 4, wherein the composition is administered to the subject before the subject is diagnosed with dental caries, carious lesions, or a combination thereof.

14. The method of claim 4, wherein the compound of formula (I) inhibits bacterial growth in the subject.

15. The method of claim 4, wherein the compound of formula (I) remineralizes a tooth of the subject.

16. The method of claim 4, wherein an effective amount of the composition is applied orally to the subject as a single treatment.

17. The method of claim 4, wherein an effective amount of the composition is applied orally to the subject as a series of treatments.

18. A kit for treating a subject with at least one of a dental caries or a carious lesion, comprising the composition of claim 3 and, optionally, a buffering agent, a preservative, or an agent for stabilizing the compound.

19. The kit of claim 18, wherein the kit is packaged into suitable packaging material and comprises instructions for administering the compound to a subject.

20. The kit of claim 19, wherein the instructions comprise one or more of paper of cardboard within the kit, label affixed to the kit, voice or video tape, or computer readable medium selected for the group consisting of disk, optical CD, DVD-ROM/RAM, Blu-Ray Disc™, electrical storage media comprising RAM and ROM, and any combination thereof.
